(19) United States
(12) Patent Application Publication
(10) Pub. No.: US 2023/0165723 A1
(43) Pub. Date: Jun. 1, 2023

(54) ADHESIVE ARTICLE AND METHOD OF MAKING SAME

(71) Applicant: KINDEVA DRUG DELIVERY L.P., Woodbury, MN (US)

(72) Inventors: Adam S. Cantor, River Falls, WI (US); Daniel H. Carlson, Arden Hills, MN (US); John R. Fenton, St Paul, WI (US); John R. Hart, St. Paul, WI (US); Glen A. Jerry, Blaine, MN (US); Gordon P. Knutson, Beldenville, MN (US); Jonathan J. O'Hare, Woodbury, MN (US); Mikhail L. Pekurovsky, Bloomington, MN (US); Timothy A. Peterson, Forest Lake, MN (US); Karl K. Stensvad, Eagan, MN (US)

(73) Assignee: KINDEVA DRUG DELIVERY L.P., Woodbury, MN (US)

(21) Appl. No.: 17/921,774

(22) PCT Filed: Apr. 27, 2021
(86) PCT No.: PCT/US2021/029354

§ 371 Date: Oct. 27, 2022

Related U.S. Application Data
(60) Provisional application No. 63/017,818, filed on Apr. 30, 2020.

(51) Int. Cl. A61F 13/00; A61F 13/02; A61K 9/70

(52) U.S. Cl. CPC A61F 13/00063; A61F 13/025; A61F 13/0253; A61F 13/0289; A61K 9/7061; A61F 2013/0071; A61F 2013/0296

(57) ABSTRACT

Various embodiments of an adhesive article and a method of forming such article are disclosed. The adhesive article includes an active pharmaceutical ingredient; a substrate having a first surface and a second surface including a first area and a second area; and a first adhesive layer disposed on the first area of the second surface of the substrate. The active pharmaceutical ingredient is present in the first adhesive layer at a first concentration. The adhesive article also includes a second adhesive layer having a first portion that is in contact with the first adhesive layer and a second portion that is disposed in the second area of the substrate. The active pharmaceutical ingredient is present in the second adhesive layer at a second concentration. The second concentration is less than the first concentration.

### CROSS-REFERENCE TO RELATED APPLICATION

This application claims the benefit of U.S. Provisional Application No. 63/017,818, filed Apr. 30, 2020, the disclosure of which is incorporated by reference herein in its entirety.

### TECHNICAL FIELD

The disclosure generally relates to adhesive articles and methods of making adhesive articles. More specifically, this disclosure relates to overcoated transdermal drug delivery devices.

### BACKGROUND

Adhesive articles such as pressure sensitive adhesive (PSA) articles typically include thin layers of pressure sensitive adhesive material that have been applied to or prepared on a substrate. Medical PSA sheets are well known and include bandages, first-aid dressings, wound care dressings, transdermal drug delivery devices, and the like. PSAs often contain ingredients in addition to a polymeric matrix that provide the basis for PSA properties. These additives include antimicrobials, antifungals, drugs, and the like that are intended to have a medicinal or therapeutic effect.

One particular type of medical sheet containing an additive is a transdermal drug delivery device. When a transdermal drug delivery device incorporates a pressure-sensitive adhesive layer including a dispersion or solution of drug and/or excipient in a polymeric matrix, the pressure sensitive adhesive article is typically prepared by coating or printing an adhesive solution or dispersion onto a substrate. That is, the adhesive polymer and the drug and/or excipient are dissolved or dispersed in a solvent, and the resulting solution or dispersion is coated onto a substrate.

### SUMMARY

In one aspect, the present disclosure provides a method of making an adhesive article. The method includes applying a first adhesive to a first area of a substrate to form a first adhesive layer, the first adhesive including an active pharmaceutical ingredient; applying a second adhesive to the first adhesive layer and a second area of the substrate to form a second adhesive layer, where the second area of the substrate completely surrounds a perimeter of the first area of the substrate. The method further includes cutting or punching a portion of the first and second adhesive layers into a shape that includes all of the first area and at least a portion of the second area surrounding the perimeter of the first area; and allowing the active pharmaceutical ingredient to diffuse from the first adhesive layer into the second adhesive layer.

In another aspect, the present disclosure provides a method of making an adhesive article. The method includes applying an active pharmaceutical ingredient to a first area of a substrate, applying an adhesive to the substrate such that the adhesive overlaps the active pharmaceutical ingredient and is in contact with the substrate in a second area of the substrate, and converting the substrate into a desired shape that includes all of the active pharmaceutical ingredient and at least a portion of the adhesive. The method further includes allowing the active pharmaceutical ingredient to diffuse into at least a portion of the adhesive.

In another aspect, the present disclosure provides a method of making adhesive articles. The method includes applying a plurality of first adhesive patches to a substrate, each of the plurality of first adhesive patches includes a first adhesive layer and an active pharmaceutical ingredient disposed within the first adhesive layer, applying a second adhesive layer to the first adhesive patches and the substrate, and forming a plurality of cutouts, each of the plurality of cutouts including one of the plurality of first adhesive patches and at least a portion of the second adhesive layer such that each cutout includes the first adhesive layer and the second adhesive layer. The method further includes allowing the active pharmaceutical ingredient to diffuse from the first adhesive layer to the second adhesive layer.

In another aspect, the present disclosure provides an adhesive article that includes an active pharmaceutical ingredient, a substrate having a first surface and a second surface, where the second surface includes a first area and a second area; and a first adhesive layer disposed on the first area of the second surface of the substrate. The first adhesive layer includes a first surface in contact with the second surface of the substrate and a second surface opposed to the second surface of the substrate. The active pharmaceutical ingredient is present in the first adhesive layer at a first concentration. The adhesive article also includes a second adhesive layer having a first surface and a second surface opposed to the first surface. A first portion of the first surface of the second adhesive layer is in contact with the second surface of the first adhesive layer, and a second portion of the first surface of the second adhesive layer is disposed on the second area of the second surface of the substrate. The active pharmaceutical ingredient in the second portion of the second adhesive layer is present at a second concentration. Further, the second concentration is less than the first concentration.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. The term “consisting of” means “including,” and is limited to whatever follows the phrase “consisting of.” Thus, the phrase “consisting of” indicates that the listed elements are required or mandatory and that no other elements may be present. The term “consisting essentially of” means including any elements listed after the phrase, and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to embodiments of the disclosure that may afford certain benefits, under certain circumstances; however, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF DRAWINGS

FIGS. 1A-C illustrate cross-sectional views of adhesive articles that include a first adhesive layer and a second adhesive layer, where FIG. 1A illustrates an adhesive article having a first thickness in a first area of a substrate of the article and a second thickness in a second area of the substrate, FIG. 1B illustrates an adhesive article having a first thickness in a first area of a substrate of the article that is the same as a second thickness in a second area of the substrate, and FIG. 1C illustrates an adhesive article having a first adhesive layer disposed on a substrate and a second adhesive layer disposed on the first adhesive layer and the substrate and extending beyond a perimeter of a desired shape of the article;

FIGS. 2A-B illustrate cross-sectional views of adhesive articles after an active pharmaceutical ingredient has been allowed to diffuse from a first adhesive layer into a second adhesive layer, where FIG. 2A illustrates an adhesive article that includes a backing that is rounded towards a substrate of the article, and FIG. 2B illustrates an adhesive article that includes a backing layer that is parallel to a substrate of the article;

FIG. 3 illustrates a top plan view of the adhesive article of FIG. 1; and

FIGS. 4-7 illustrate one technique of making adhesive articles, where FIG. 4 illustrates applying a plurality of first adhesive patches to a substrate; FIG. 5 illustrates applying a second adhesive to the first adhesive patches and the substrate; FIG. 6 illustrates forming a plurality of cutouts; and FIG. 7 illustrates allowing an active pharmaceutical ingredient to diffuse from a first adhesive layer of the first adhesive patches to a second adhesive layer.

### DETAILED DESCRIPTION

The present disclosure provides an adhesive article and a method of forming the adhesive article. In one or more embodiments, the adhesive article is a drug delivery device.

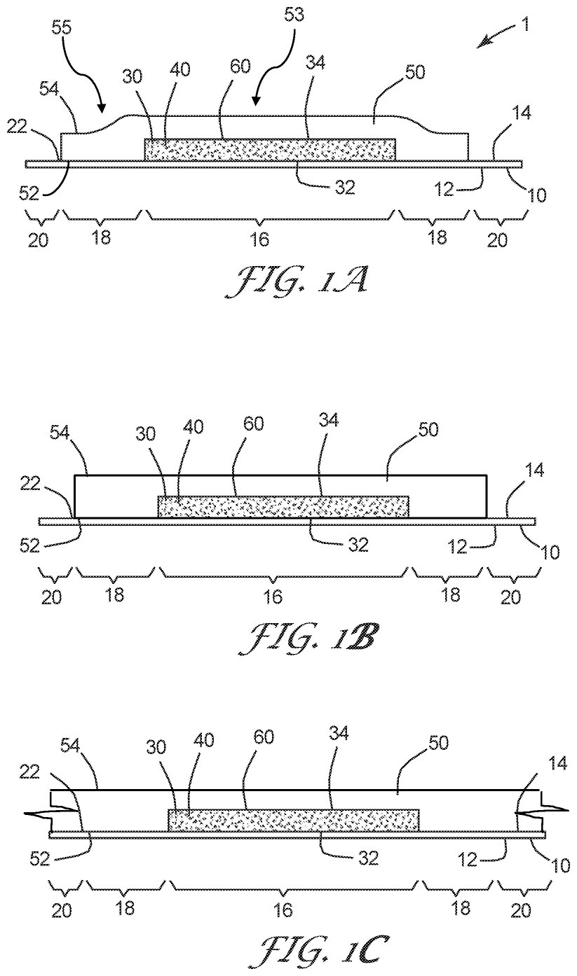

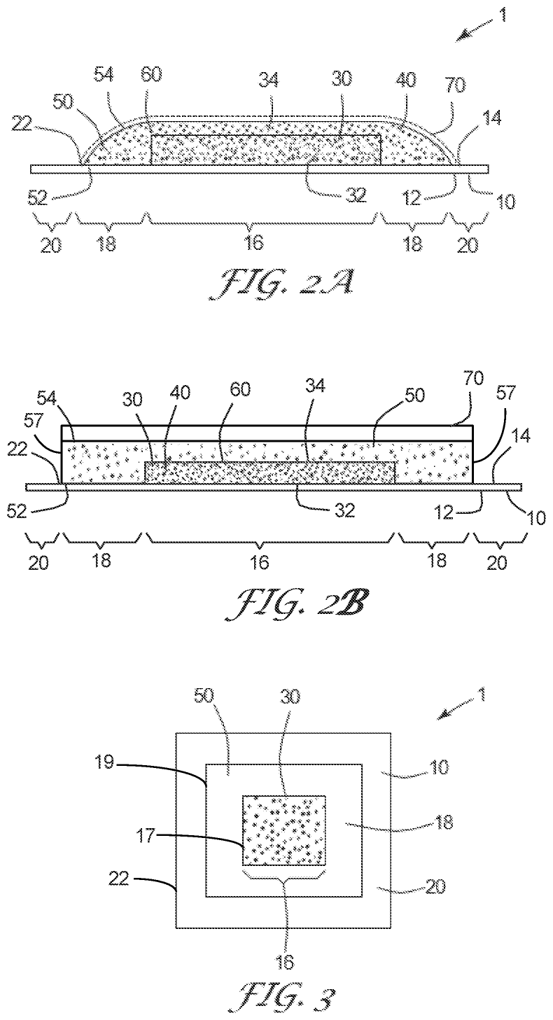

FIGS. 1-2 illustrate various cross sectional views of an adhesive article 1 that includes a substrate 10 having a first surface 12 and a second surface 14. The second surface 14 of the substrate 10 includes at least a first area 16 and a second area 18. The first area 16 may form various shapes or patterns, including circular, ovular, square, rectangular, heart-shaped, or any other shape suitable for application to the skin. In one or more embodiments, the second area 18 surrounds a perimeter 17 of the first area 16 as can be seen in FIG. 3, which is a plan view of the adhesive article of FIG. 1. The outer shape of the second area 18 may form various shapes or patterns, including circular, ovular, square, rectangular, heart-shaped, or any other shape suitable for application to the skin.

Referring to FIG. 1A, a first adhesive layer 30 is disposed on the first area 16 of the second surface 14 of the substrate 10 and includes a first surface 32 and a second surface 34. The first surface 32 of the first adhesive layer 30 is in contact with the second surface 14 of the substrate 10, and the second surface 34 of the first adhesive layer is opposed to the second surface of the substrate. The first adhesive layer 30 also includes a diffusible molecule that may preferably be a therapeutically active pharmaceutical ingredient 40. The adhesive article 1 also includes a second adhesive layer 50 that includes a first surface 52 and a second surface 54 opposed to the first surface. A first portion 53 of the first surface 52 of the second adhesive layer 50 is in contact with the second surface 34 of the first adhesive layer 30, and a second portion 55 of the first surface 52 of the second adhesive layer is disposed on the second area 18 of the second surface 14 of the substrate 10. As such, the second area 18 of the substrate 10 may define an area between the perimeter 17 of the first area 16 or first adhesive layer 30 and a perimeter 19 (FIG. 3) of the second adhesive layer 50, or in other embodiments a perimeter of the PSA article 1. Beyond the perimeter 19 of the second adhesive layer 50 may be a third area 20 of the substrate 10. The third area 20 is generally adjacent to the second area 18 and remains uncovered from the first or second adhesive layers 30, 50, i.e., the third area is free of the first and second adhesive layers. The division between the second area 18 and third area 20 may define a desired shape of the PSA article 1. In one or more embodiments, the third area 20 defines a perimeter 22 of the adhesive article 1. As discussed in more detail herein, the second adhesive layer 50 may be initially applied so that it extends beyond perimeter 22 (as shown in FIG. 1C) and then the portion of second adhesive layer extending beyond perimeter 22 is removed before the active pharmaceutical ingredient 40 in the first adhesive layer 30 can diffuse beyond the perimeter 22.

When the second adhesive layer 50 is applied to the substrate 10, there may be superficial topography differences between the first area 16 and the second area 18 as shown in FIG. 1A. This can be due to the presence of the first and second adhesive layers 30, 50 being disposed in the first area 16, while only the second adhesive layer 50 is present in the second area 18. Therefore, the adhesive article 1 may have a first thickness proximate the first area 16 and a second thickness proximate the second area 18 as measured in a direction orthogonal to the second surface 14 of the substrate 10. In one or more embodiments, the first thickness is less than the second thickness. However, as time progresses after the application of the second adhesive layer 50 the difference in thickness between the first area 16 and the second area 18 may become smaller due to rheological deformation and/or relaxation (e.g., creep) of the first adhesive layer 30 and/or second adhesive layer 50. In one or more embodiments, the second adhesive layer 50 may be applied in such a manner as to have a consistent (i.e., flat) topography as shown in FIG. 1B along the top surface 54.

As will be discussed in greater detail herein, a conversion process includes cutting the PSA article 1 into a desired shape. That is, the second adhesive layer 50 may initially be applied such that it extends beyond the perimeter 22 as shown in FIG. 1C. The inclusion of the second adhesive layer 50 enables a PSA article 1 to be formed in a desired shape while eliminating or minimizing loss of any active pharmaceutical ingredient 40, thereby significantly reducing the cost of production. Therefore, the conversion process can be carried out before the active pharmaceutical ingredient 40 can diffuse beyond the perimeter 22 of the desired shape. As such, the PSA article 1 includes the adhesive layers 30, 50 converted into the desired shape along with the substrate 10, which can be a release liner. A desired shape of the PSA article 1 may include, among other shapes, circular, ovular, rectangular, heart-shaped, or any other shape that can adhere to a surface of the skin.

Referring to FIG. 2A, the first adhesive layer 30 and second adhesive layer 50 may form an interface 60 between the first surface 52 of the second adhesive layer 50 and the second surface 34 of the first adhesive layer 30. Adhesive polymer chains from each of the first and second adhesive layers 30, 50 may become entangled with each other at the interface 60 as time progresses, such that the interface 60 is blurred or becomes essentially indiscernible, and the first and second adhesive layers 30, 50 form what appears to be a single homogenous adhesive. In one or more embodiments, the adhesive polymer chains of the first adhesive layer 30 and second adhesive layer 50 may be thermodynamically incompatible to the extent that no or little entanglement occurs at the interface 60. Regardless of whether entanglement occurs, the first and second adhesive layers 30, 50 are in diffusional communication as the active pharmaceutical ingredient 40 may diffuse from the first adhesive layer to the second adhesive layer. In one or more embodiments, conversion of the PSA article 1 occurs prior to any substantial amount of diffusion of the active pharmaceutical ingredient 40 from the first adhesive layer 30 to the second adhesive layer 50. In one or more embodiments, the conversion process may occur at any time prior to the diffusion of the active pharmaceutical ingredient 40 beyond the perimeter 22 of the desired shape.

As shown in FIGS. 2A-B, diffusion of the active pharmaceutical ingredient 40 occurs both vertically and horizontally with respect to the first and second adhesive layers 30, 50. Thus, the active pharmaceutical ingredient 40 diffuses perpendicular to the substrate 10 into the second adhesive layer 50 and towards the second surface 54 of the second adhesive layer from the first adhesive layer 30. Additionally, the active pharmaceutical ingredient 40 diffuses outwardly, or parallel to the substrate 10, into the second adhesive layer 50 towards the perimeter 22 of the PSA article 1. The concentration gradient of the active pharmaceutical ingredient 40 between the first adhesive layer 30 and second adhesive layer 50 in the vertical direction proximate the first area 16 may differ from the concentration gradient between the first adhesive layer and the second adhesive layer proximate the perimeter 22 of the PSA article 1. As such, the PSA article 1 may have a first concentration gradient of active pharmaceutical ingredient 40 proximate the first area 16 and a second concentration gradient proximate the second area 18. The first concentration gradient may be less than the second concentration gradient. For instance, diffusion of the active pharmaceutical ingredient 40 to the perimeter 22 of the PSA article 1 may lead to a concentration of active pharmaceutical ingredient 40 in the second adhesive layer 50 proximate the second area 18 that is less than 10% of the concentration of active pharmaceutical ingredient 40 in the first adhesive layer proximate the second area of the PSA article 1. In another embodiment, diffusion of the active pharmaceutical ingredient 40 to the perimeter 22 of the PSA article 1 may lead to a concentration of active pharmaceutical ingredient in the second adhesive layer 50 proximate the second area 18 that is less than 50% of the concentration of active pharmaceutical ingredient 40 in the second adhesive layer 50 proximate the second area 18 of the PSA article. In yet another embodiment, diffusion of the active pharmaceutical ingredient 40 to the perimeter 22 of the PSA article 1 may lead to a concentration of active pharmaceutical ingredient in the second adhesive layer 50 proximate the second area 18 that is less than 90% of the concentration of active pharmaceutical ingredient 40 in the first adhesive layer 30 proximate the second area 18 of the PSA article 1. In yet another embodiment, diffusion of the active pharmaceutical ingredient 40 to the perimeter 22 of the PSA article 1 may lead to a concentration of active pharmaceutical ingredient 40 in the second adhesive layer 50 proximate the second area 18 that is more than 95% and less than 100% of the concentration of active pharmaceutical ingredient in the second adhesive layer 50 proximate the second area 18 of the PSA article 1.

Additionally, the PSA article 1 may include a thermodynamic activity gradient between the first and second adhesive layers 30, 50 that affects the diffusion of the diffusible active pharmaceutical ingredient 40 from the first adhesive layer 30 to the second adhesive layer 50. If the polymer used in the first adhesive layer 30 is composed of a different composition than the polymer used in the second adhesive layer 50, the diffusion of the active pharmaceutical ingredient 40 may form the thermodynamic activity gradient relative to the differing concentrations of active pharmaceutical ingredient between the first and second adhesive layers 30, 50. In particular, the active pharmaceutical ingredient 40 may have a different solubility in the polymers of the first adhesive layer 30 and second adhesive layer 50 and thus the concentration at, or near to, thermodynamic equilibrium of the active pharmaceutical ingredient may differ between the first adhesive layer and second adhesive layer.

As is further shown in FIGS. 2A-B, the PSA article 1 may include a backing layer 70 in contact with the second surface 54 of the second adhesive layer 50. The backing layer 70 may be applied prior to or after the converting process. As shown in FIG. 2A, outer edges 72 of the backing layer 70 may round down towards the substrate 10. In one or more embodiments, as shown in FIG. 2B, the backing layer 70 may be parallel to the substrate 10 and an outer edge 57 of the second adhesive layer 50 may be exposed.

FIG. 3 illustrates a top plan view of the PSA article 1 after the second adhesive layer 50 has been applied to the substrate 10. As shown, the first area 16 of the substrate 10 includes an area of the substrate 10 covered by the first adhesive layer 30. The second area 18 of the substrate 10 includes an area surrounding the first area 16, where the second adhesive layer 50 is in direct contact with the substrate 10. The third area 20 of the substrate 10 is shown as an area of the substrate 10 beyond the second adhesive layer 50, where the second surface 14 of the substrate 10 is exposed. Any suitable backing material (not shown), e.g., backing layer 70, may be disposed on the second surface 54 of the second adhesive layer 50 to form a finished drug delivery device 1.

FIGS. 4-7 illustrate adhesive articles at various stages of a method of manufacturing. In FIG. 4, the first adhesive layer 30 is disposed on the substrate 10 (e.g., the second surface 14 of the substrate). The first adhesive layer 30 may be applied as a plurality of patches or printouts 30a disposed on the substrate 10. Individually, each first adhesive patch 30a encompasses the first area 16 of the substrate 10 of each drug delivery device 1 as shown in FIGS. 1A-C and 3. In one or more embodiments, each patch 30a has a surface area of between 5 and 100 cm², 5 and 40 cm², and 5 and 20 cm².

Prior to application of the first adhesive layer 30 to the substrate 10, the active pharmaceutical ingredient 40 may be mixed in with an adhesive polymer to form a liquid adhesive coating composition. The active pharmaceutical ingredient 40 may also be mixed with other solids or liquids to prepare the liquid coating composition by any conventional method known to those skilled in the art. In particular, a solid or liquid active pharmaceutical ingredient may be dissolved or dispersed in a liquid or mixture of liquids to prepare a liquid adhesive coating composition. By mixing the active pharmaceutical ingredient with an adhesive polymer to prepare a liquid adhesive coating composition and precisely coating a pre-metered amount of the liquid adhesive coating composition to the substrate 10, the method ensures a consistent dosage of an active pharmaceutical ingredient for each patch.

The first adhesive layer 30 may be applied via patch, or intermittent, coating to form discrete areas of coating (i.e., a plurality of patches 30a). In one or more embodiments, the first adhesive layer 30 may be applied via slot coating to form a plurality of patches 30a. Additionally, the coating head may be any suitable coating head, e.g., at least one of a single slot die, a multiple slot die, a single orifice die, or a multiple orifice die. In one or more embodiments, the coating head is a single slot die having a single die slot, where the external opening includes the die slot. In one or more embodiments, the geometry of the single slot die is selected from a sharp-lipped extrusion slot die, a slot fed knife die with a land, or a notched slot die. In any of the foregoing exemplary embodiments of coating processes, the source of the first coating liquid may include a pre-metered coating liquid delivery system selected from a syringe pump, a dosing pump, a gear pump, a servo-driven positive displacement pump, a rod-driven positive displacement pump, or a combination thereof. In one or more embodiments, the discrete areas of coating have a surface area of between 1 and 100 cm², 5 and 100 cm², 5 and 40 cm², and 5 and 20 cm².

In one or more embodiments, printing methods may be used to apply the first adhesive layer 30 to the substrate 10, including non-contact printing, flexographic, gravure, or inkjet printing, spray coatings, or others known to those of skill in the art. A printer will include one or more fluid dispensing mechanisms and a substrate handling system designed to receive and present the substrate 10 to the fluid dispensing mechanism. The fluid dispensing mechanisms used for non-contact printing may include ink-jetting, stream-jetting, ultrasonic atomization, and the like. Combinations of each of these fluid dispensing mechanisms may also be used to achieve desired dispensing rates and volumes. These techniques also allow for precision printing of fluids in high resolution patterns to form the desired shape of the patch 30a.

FIG. 5 illustrates another step of the method, which includes applying the second adhesive layer 50 to the first adhesive patches 30a and the substrate 10. The second adhesive layer 50 does not initially include the active pharmaceutical ingredient. The second adhesive layer 50 is overcoated on top of each of the plurality of first adhesive patches 30a and at least a portion of the substrate 10. Coating or printing methods described herein for the first adhesive layer 30 may likewise be used to apply the second adhesive layer 50. The second adhesive layer 50 overlaps the first adhesive layer 30 proximate the first area 16 and is disposed in the second area 18 (See FIGS. 1 and 2).

In one or more embodiments, the method may include drying (i.e., removal of carrier solvent(s)) the first adhesive layer 30 prior to application of the second adhesive layer 50. In one embodiment, drying of the first adhesive layer 30 prior to the application of the second adhesive layer 50 does not result in a loss of the active pharmaceutical ingredient 40.

In one or more embodiments, the second adhesive layer 50 may be applied prior to drying (i.e., removal of carrier solvent(s)) of the first adhesive layer 30. The combination of the first and second adhesive layers 30, 50 is then dried after application of the second adhesive layer 50. In one or more embodiments, simultaneous drying of the first and second adhesive layers 30, 50 does not result in a loss of active pharmaceutical ingredient 40.

In yet another embodiment, the backing layer 70 (not shown) may be disposed adjacent to the second adhesive layer 50 such that the first and second adhesive layers 30, 50 are disposed between the backing layer and the substrate 10. In one or more embodiments, the backing layer 70 is in contact with the second surface 54 of the second adhesive layer 50. The backing layer 70 may be applied prior to the conversion process and may accommodate the desired shape of the adhesives. However, the backing layer 70 may also be applied after the conversion process has been completed. In this embodiment, the conversion process (as discussed herein) is carried out to form the desired shape of the adhesive. After conversion the backing layer 70 may then be applied to the second surface 54 of the second adhesive layer 50.

FIG. 6 illustrates a plurality of adhesive cutouts 50a on the substrate 10 after the adhesive articles have been converted to desired shapes. Each of the plurality of adhesive cutouts 50a includes one of the plurality of first adhesive patches 30a and at least a portion of the second adhesive layer 50 such that each cutout includes a first adhesive layer 30 and a second adhesive layer 50. The conversion process includes cutting through the first and second adhesive layers 30, 50 at the perimeter 22 of the desired shape of each adhesive article 1. In one or more embodiments, the excess of second adhesive layer 50 that is present in the third area 20 may be removed from the substrate 10 once the adhesive articles 1 have been converted. In this embodiment, conversion of the adhesive articles 1 includes cutting through the first and second adhesive layers 30, 50 to form the plurality of adhesive cutouts 50a on the surface of the substrate 10. Further, the substrate 10 remains intact and the remaining or excess second adhesive layer 50 is peeled from the second surface 14 of the substrate 10 to leave the plurality of patches each defined by the perimeter of an individual adhesive article 1. As shown schematically in FIG. 6, the active pharmaceutical ingredient 40 (represented by the shading) is present in the first adhesive layer 30, but the conversion of the outer shape of the second adhesive layer 50 takes place before any appreciable amount of active pharmaceutical ingredient has diffused into the second adhesive layer. In one or more embodiments, the conversion process includes cutting through the first and second adhesive layers 30, 50, as well as the substrate 10 to form individual adhesive articles 1 (not shown). In one or more embodiments, the remaining substrate 10 and excess second adhesive layer 50 is discarded, and there is no need to peel the waste or excess second adhesive layer from the substrate 10. A backing layer may then be applied to the individual adhesive articles 1. In one or more embodiments, the adhesive articles 1 each have a surface area of between 5 and 100 cm², 5 and 40 cm², and 5 and 20 cm².

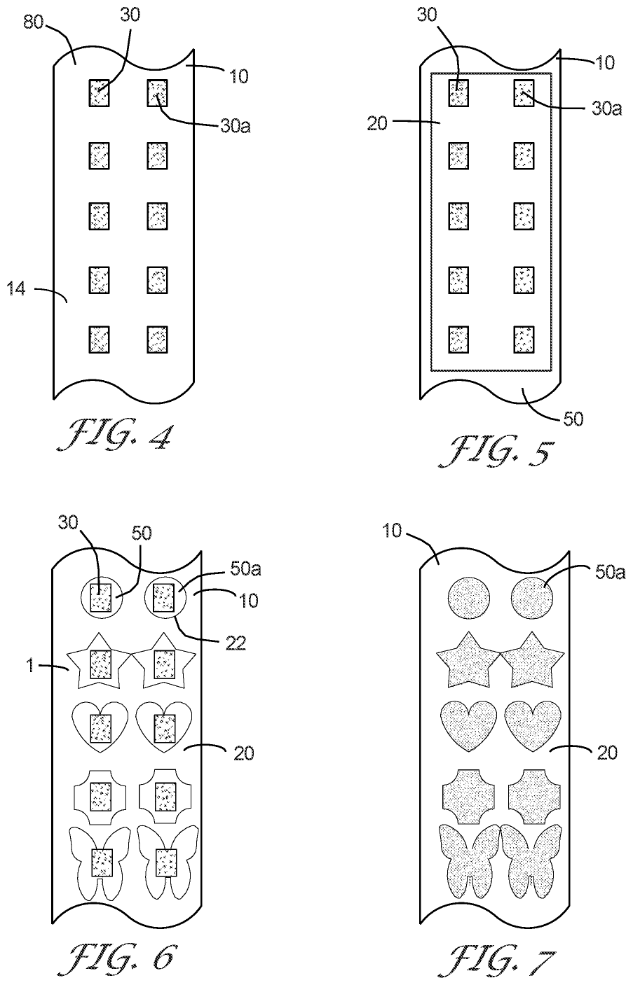

In FIG. 7, the first and second adhesive layers 30, 50 are in contact with each other, and a portion of the active pharmaceutical ingredient 40 has diffused from the first adhesive layer to the second adhesive layer, as represented by the more complete and more uniform shading of the second adhesive layer. The process of converting the adhesive article 1 into a desired shape can be carried out before diffusion of the active pharmaceutical ingredient 40 to a portion of the second adhesive layer 50 beyond the perimeter of the desired shape of the drug delivery device 1 can take place. The rate of diffusion of the active pharmaceutical ingredient 40 depends not only on the characteristics of the active pharmaceutical ingredient 40 but the characteristics of both the first and second adhesive layers 30, 50 as well. For instance, the active pharmaceutical ingredient 40 can, in one or more embodiments, have a low molecular weight enabling diffusion through the adhesive layers 30, 50. Diffusion may be aided by the addition of various excipients that enhance the diffusion rate of the active pharmaceutical ingredient 1 throughout the drug delivery device 1. Various factors that may control the diffusion of the active pharmaceutical ingredient can be controlled to time the addition of the second adhesive layer and the conversion of the adhesive article 1 into a desired shape to avoid loss of active pharmaceutical ingredient. As such, the process of converting the adhesive articles into a desired shape can be carried out with minimal or no loss of any active pharmaceutical ingredient. Limiting the loss of active pharmaceutical ingredient can reduce costs and increase the accuracy of pre-metered doses of the active pharmaceutical ingredient 40 in the drug delivery device 1. Thus, the various methods described herein can provide an effective process for adhesive articles 1 having various shapes and doses of active pharmaceutical ingredients, while also limiting the cost and waste of manufacturing.

The active pharmaceutical ingredient 40 is intended to have a medicinal or therapeutic effect. This active pharmaceutical ingredient may be a drug, antimicrobial agent, antifungal agent, cosmetic agent, or pharmaceutically effective excipient.

In one or more embodiments, the active pharmaceutical ingredient is a drug. In one or more embodiments, the adhesive article includes a transdermal drug delivery composition. The drug can be present in an amount such that the composition delivers a therapeutically effective amount for the condition being treated. This amount will vary according to the type of drug used, the condition to be treated, the amount of time the composition is allowed to remain in contact with the skin of the subject, and other factors known to those of skill in the art. However, the amount of drug present in the transdermal drug delivery composition of the adhesive device will generally be about 0.01 to 40 wt-%, preferably about 1.0 to 20 wt-%, based on the total weight of the composition. In a composition of the disclosure the drug is dispersed or dissolved in a pressure sensitive adhesive. Further, the active pharmaceutical ingredient can have a low molecular weight. Therefore, the active pharmaceutical ingredient may have a molecular weight below 1,000 Daltons in some embodiments of the disclosure. In one or more embodiments, the molecular weight of the active pharmaceutical ingredient may be between 100 and about 600 Daltons. In one or more embodiments, the molecular weight of the active pharmaceutical ingredient may be between about 100 and 500 Daltons.

Any drug that is suitable for transdermal delivery may be used in the various embodiments of adhesive articles described herein. Examples of useful drugs include, but are not limited to, anti-inflammatory drugs, both steroidal (e.g., hydrocortisone, prednisolone, triamcinolone) and nonsteroidal (e.g., naproxen, piroxicam); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotozoals (e.g., metronidazole); antifungals (e.g., nystatin); coronary vasodilators (e.g., nitroglycerin); calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors (e.g., zileuton), and angiotensin converting enzyme inhibitors (e.g., captopril, lisinopril); other antihypertensives (e.g., propranolol); leukotriene antagonists; antiulceratives such as H2 antagonists; steroidal hormones (e.g., progesterone, testosterone, estradiol); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinoline-4-amine, and other compounds disclosed in U.S. Pat. No. 4,689,338, incorporated herein by reference, acyclovir); local anesthetics (e.g., benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl); peptide hormones (e.g., human or animal growth hormones, LHRH); sex hormones (e.g., estrogens, testosterone, progestins such as levonorgestrel, norethindrone, gestodene); cardioactive products such as atriopeptides; proteinaceous products (e.g., insulin); enzymes (e.g., antiplaque enzymes, lysozyme, dextranase); antinauseants (e.g., scopolomine); anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatriptan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; and the like, as well as pharmaceutically acceptable salts and esters thereof.

In one or more embodiments, the active pharmaceutical ingredient may be an antimicrobial agent. Antimicrobial agents may include any broad-spectrum antimicrobial agent that is suitable for topical application. Examples of suitable antimicrobial agents are iodine, including iodine complexes with sodium or potassium iodide as well as polymeric complexes often called iodophors such as povidone-iodine and polyethylene glycol complexes, hexylresorcinol, chlorhexidine or a suitable salt thereof such as chlorhexidine gluconate or chlorhexidine acetate, triclosan, p-chloro-m-xylenol (PCMX), phenols, peroxides, silver and silver salts such as silver chloride, silver oxide and silver sulfadiazine, long chain alkyl quaternary ammonium compounds, and mono C₈-C₁₂ alkyl esters of glycerin and propylene glycol. Antifungal agents may also be incorporated and include any of the “azoles” such as miconazole nitrate, chlortrimazole, econazole, ketoconizole and the like as well as tolnaftate and undecylic acid and its salts. Iodine, iodine complexes with sodium or potassium iodide, povidone-iodine, and chlorhexidine are preferred antimicrobial agents.

In one or more embodiments, the adhesive article may include an excipient. These excipients may include skin penetration enhancers or solubilizers in transdermal drug delivery systems. Exemplary materials include C8-C36 fatty acids such as isostearic acid, octanoic acid, and oleic acid; C8-C36 fatty alcohols such as oleyl alcohol and lauryl alcohol; lower alkyl esters of C8-C36 fatty acids such as ethyl oleate, isopropyl myristate, butyl stearate, and methyl laurate; di(lower) alkyl esters of C6-C8 diacids such as diisopropyl adipate; monoglycerides of C8-C36 fatty acids such as glyceryl monolaurate; tetraglycol (tetrahydrofurfuryl alcohol polyethylene glycol ether); tetraethylene glycol (ethanol,2,2′-(oxybis(ethylenoxy))diglycol); C6-C36 alkyl pyrrolidone carboxylates; polyethylene glycol; propylene glycol; 2-(2-ethoxyethoxy)ethanol; diethylene glycol monomethyl ether; N,N-dimethyldodecylamine-N-oxide and combinations of the foregoing. Alkylaryl ethers of polyethylene oxide, polyethylene oxide monomethyl ethers, and polyethylene oxide dimethyl ethers are also suitable, as are solubilizers such as glycerol and N-methyl pyrrolidone. The terpenes are another useful class of excipients, including pinene, d-limonene, carene, terpineol, terpinen-4-ol, carveol, carvone, pulegone, piperitone, menthone, menthol, neomenthol, thymol, camphor, bomeol, citral, ionone, and cineole, alone or in any combination. Of the terpenes, terpineol, particularly α-terpineol, is preferred.

Preferred excipients include glyceryl monolaurate, terpineol, lauryl alcohol, tetraglycol, tetraethylene glycol, propylene glycol, isopropyl myristate, ethyl oleate, methyl laurate, and 2-(2-ethoxyethoxy)ethanol.

While many of the excipients enumerated herein are known to affect skin penetration rate, certain excipients affect aspects of performance other than and in addition to skin penetration rate. For example, such excipients are useful in softening or increasing the compliance value and/or lowering the glass transition temperature of polymers, such that the resulting composition is more suitable for use as a pressure sensitive adhesive. The excipients may also increase the rate of diffusion between the first and second adhesives after the second adhesive has been applied to the substrate.

In a composition of the disclosure the excipient(s) is dispersed. In one or more embodiments, the excipient is substantially uniformly dispersed. Further, in one or more embodiments, the excipient is dissolved in the composition. Where the excipient is a penetration enhancer, it is present in an amount that enhances drug permeation through the skin compared to a like composition not containing the penetration enhancer(s) when this phenomenon is measured using a standard skin penetration model, such as in U.S. Pat. No. 5,585,111 (Peterson), the disclosure of which is herein incorporated by reference. The total amount of excipient will generally be about 0.1 to about 40% by weight based on the total weight of the composition.

A dye may be included in the first or second adhesive layers. The dye may be of similar molecular weight and have similar properties to that of the diffusible molecule to aid in the detection of diffusion of the diffusible molecule. In one or more embodiments, the dye may be a low molecular weight UV or fluorescent detectable dye.

Acrylate polymers and copolymers can be constituents of the first and/or second adhesive layers of the adhesive article. Suitable acrylate copolymers can include one or more A monomers such as alkyl (meth)acrylates or aromatic or cycloalkyl (meth)acrylates. Useful alkyl acrylates include monofunctional unsaturated monomers such as acrylate esters of non-tertiary alkyl alcohols, the alkyl groups containing 1 to 18 carbon atoms in the alkyl group, preferably about 4 to about 12 carbon atoms, and mixtures thereof. Examples of suitable alkyl acrylates include methyl, ethyl, n-butyl, n-pentyl, n-hexyl, isoheptyl, n-nonyl, n-decyl, isohexyl, isodecyl, isooctyl and 2-ethylhexyl acrylates. Preferred alkyl acrylates include isooctyl acrylate, 2-ethylhexyl acrylate, and n-butyl acrylate. Useful alkyl methacrylates include monofunctional unsaturated monomers such as methacrylate esters of non-tertiary alkyl alcohols, the alkyl groups containing 1 to 18 carbon atoms in the alkyl group, preferably 1 to about 12 carbon atoms, and mixtures thereof. Examples of suitable alkyl methacrylates include methyl methacrylate, ethyl methacrylate, n-butyl methacrylate, n-hexyl methacrylate, noctyl methacrylate, isooctyl methacrylate and decyl methacrylate. Examples of suitable aromatic monomers and normal or bridged cycloalkyl (meth)acrylates include isobomyl acrylate, isobomyl methacrylate, 4-t-butylcyclohexyl methacrylate, cyclohexyl acrylate, cyclohexyl methacrylate, phenyl acrylate, 2-naphthyl acrylate, and 2-naphthyl methacrylate. Acrylate copolymers can include about 5 to about 95 percent by weight, more preferably 55 to 95 percent by weight, based on the total weight of all monomers in the copolymer, of one or more A monomers. In another embodiment, silicone polymers, block copolymer synthetic rubbers, polyisobutane polymers, other polymers suitable for transdermal drug delivery pressure-sensitive adhesives may be used to form a portion of the first and/or second adhesive layers. Mixtures of the aforementioned polymers may also be suitable for use in either the first and/or second adhesive layers.

Acrylate copolymers may optionally further include one or more B monomers. Suitable B monomers include those containing a functional group such as carboxylic acid, sulfonamide, urea, carbamate, carboxamide, hydroxy, amino, oxy, and cyano. Exemplary B monomers include acrylic acid, methacrylic acid, maleic acid, a hydroxyalkyl acrylate containing 2 to 4 carbon atoms in the hydroxyalkyl group, a hydroxyalkyl methacrylate containing 2 to 4 carbon atoms in the hydroxyalkyl group, acrylamide, methacrylamide, an alkyl substituted acrylamide containing 1 to 8 carbon atoms in the alkyl group, N-vinyl formamide, N-vinyl acetamide, N-vinyl valerolactam, N-vinyl caprolactam, N-vinyl-2-pyrrolidone, glycidyl methacrylate, vinyl acetate, alkoxyethyl acrylate containing 1 to 4 carbon atoms in the alkoxy group, alkoxyethyl methacrylate containing 1 to 4 carbon atoms in the alkoxy group, 2-ethoxyethoxyethyl acrylate, furfuryl acrylate, furfuryl methacrylate, tetrahydrofurfuryl acrylate, tetrahydrofurfuryl methacrylate, propylene glycol monomethacrylate, propylene oxide methyl ether acrylate, acrylonitrile, methacrylonitrile, di(lower alkyl)amino ethyl acrylate, di(lower alkyl)amino ethyl methacrylate, and di(lower alkyl)aminopropyl methacrylamide, where (lower alkyl) in the preceding examples represents alkyl groups containing 1 to 4 carbon atoms. Preferred B monomers include acrylic acid, methacrylic acid, acrylamide, 2-hydroxy ethyl acrylate, and vinyl acetate. Acrylate copolymers can include about 5 to about 55 percent by weight, more preferably about 5 to about 45 percent by weight, based on the total weight of all monomers in the copolymer, of one or more B monomers.

Acrylate copolymers may optionally further include a substantially linear macromonomer copolymerizable with the A and B monomers and having a weight average molecular weight in the range of about 500 to about 100,000, preferably about 2,000 to about 50,000 and more preferably about 5,000 to about 30,000. The macromonomer, when used, is generally present in an amount of not more than about 20%, preferably not more than about 10% by weight based on the total weight of all monomers in the copolymer. Suitable macromonomers include polymethylmethacrylate, styrene/acrylonitrile copolymer, polyether, and polystyrene macromonomers. Examples of useful macromonomers and their preparation are described in Krampe et al., U.S. Pat. No. 4,693,776, the disclosure of which is incorporated herein by reference.

The adhesive layers of the present disclosure may have varying adhesive properties depending on the types and amounts of polymers used, types and amounts of any other additives, and any other processing treatment, such as curing, applied to the adhesive layers.

In one or more embodiments, the polymer of the both the first and second adhesive layers are pressure sensitive adhesives, preferably an acrylate copolymer pressure sensitive adhesive, more preferably having an inherent viscosity in the range of about 0.2 dl/g to about 2 dl/g, more preferably about 0.5 dl/g to about 1.6 dl/g. In one or more embodiments, the polymer of the second adhesive is the same as the polymer of the first adhesive to ensure uniform or near uniform distribution of the active pharmaceutical ingredient in the drug delivery device at thermodynamic equilibrium.

The first and second adhesive layers can be applied to any suitable substrate that can be a sheet material or a shaped article. In one or more embodiments, the substrate includes a release liner. Suitable release liners are discussed further herein. In one or more embodiments, the substrate may include a flexible material and a medical adhesive, which may be a medical PSA sheet. The adhesive layer polymers of the present disclosure can be easily coated upon suitable flexible or inflexible substrates by conventional coating techniques, such as roll coating, spray coating, curtain coating, and the like to produce coated polymeric sheet materials in accord with the present disclosure. The adhesive layer polymers may also be coated without modification by extrusion coating, coextrusion, hot-melt coating, and the like by employing suitable conventional coating devices for this purpose. The adhesive layer polymers may also be prepared in place on the substrate, for example using the photopolymerization method described herein. The adhesive layer polymers may be optionally coated as discontinuous patterns using conventional printing methods, such as flexographic printing or screen printing. In one or more embodiments, the use of intermittent slot die coating is used to apply the first adhesive to the substrate. The second adhesive layer may preferably be applied via self-metered coating processes including notch bar coaters, knife coaters, roll coaters or similar coating methods of pre-metered coating.

Sheet materials can be provided as individual pieces or as a continuous web, for example in a rollstock form. Exemplary substrates can be made from any material conventionally utilized as a tape backing, release liner, or any other flexible material. Typical examples of flexible backing materials employed as conventional tape backings that may be useful for the present disclosure include those made of paper, plastic films such as polypropylene, polyethylene, particularly low density polyethylene, linear low density polyethylene, metallocene polyethylenes, high density polyethylene, polyvinyl chloride, polyester (e.g., polyethylene terephthalate), ethylene-vinyl acetate copolymer, polyurethane, cellulose acetate and ethyl cellulose. Backings that are layered such as polyethylene terephthalate-aluminum-polyethylene composites are also suitable.

Backings may also be prepared of fabric such as woven fabric formed of threads of synthetic or natural materials such as cotton, nylon, rayon, glass, ceramic material, and the like or nonwoven fabric such as air laid webs of natural or synthetic fibers or blends of these.

Suitable substrates or release liners include conventional release liners including a known sheet material such as a polyester web, a polyethylene web, a polypropylene web, or a polyethylene-coated paper coated with a suitable fluoropolymer or silicone-based coating.

### EXAMPLES

### Example 1

An “Adhesive Formulation A” of isooctyl acrylate/acrylamide/vinyl acetate (75/5/20) copolymer (32 weight % solids in ethyl acetate/methanol) was prepared according to the procedure described for “Copolymer B” in U.S. Pat. No. 9,375,510. An “Adhesive Formulation B” was prepared according to “Adhesive Formulation A” except for the addition of TINOPAL OB Optical Brightener (a 2,5-thiophenediylbis(5-tert-butyl-1,3-benzoxazole) fluorescent compound, obtained from the BASF Corporation, Florham Park, N.J.) into the adhesive at 0.1% by weight. The compound was mixed into the adhesive using a LabRAM mixer (Resodyn Corporation, Butte, Mont.) operated for about 2 minutes at 100% intensity. Based on a molecular weight of 430.6 g/mole, TINOPAL OB Optical Brightener was used to provide a conservative representation of diffusion through the dry adhesive by a transdermal active pharmaceutical ingredient (API) such as fentanyl, which has a molecular weight of 336.5 g/mol.

An adhesive article 1 was prepared as shown in FIG. 3. A sheet of siliconized PET release liner was used as the substrate 10. A frame shaped mask with a rectangular opening was formed by placing a die cut piece of 3M General Purpose PTFE Skived Film Tape 5180 (3.5 mil thickness, obtained from the 3M Corporation, St. Paul, Minn.) over the substrate. The exposed surface of the substrate defined by the mask was coated with “Adhesive Formulation B” using a benchtop notch bar coater with the mask providing the coating gap. The adhesive was allowed to air dry and then the mask was removed leaving a patch of a first adhesive layer 30 containing the added TINOPAL OB Optical Brightener.

Next, the benchtop notch bar coater (gap setting of 0.008 inches (0.2 mm)) was used to coat “Adhesive Formulation A” over the dried adhesive patch. The coating of “Adhesive Formulation A” as a second adhesive layer 50 completely covered the surface of the dried adhesive patch as well as an area of the substrate beyond the perimeter of the dried adhesive patch. The second coating was allowed to air dry.

The resulting finished article (1) was observed for fluorescence for ten days using 365 nm light. Over the course of ten days, the fluorescence signature from TINOPAL OB Optical Brightener was observed to diffuse laterally into the second coated region and toward the outer perimeter of the adhesive article.

### Example 2

An “Adhesive Formulation C” is prepared according to the procedure described for “Adhesive Formulation A” in Example 1 except that an active pharmaceutical ingredient (API) selected from fentanyl, buprenorphine, rivastigmine, lidocaine, clonidine, etidocaine, estradiol, testosterone or clobetasol is mixed into the adhesive at a concentration of about 0.0001% to about 20% by weight using a LabRAM mixer (Resodyn Corporation) operated for about 2 minutes at 100% intensity. Each “Adhesive Formulation C” that is prepared contains a single API selected from the API list above.

A drug delivery device 1 is prepared as shown in FIG. 3. A sheet of siliconized PET release liner is used as the substrate 10. A frame shaped mask with a rectangular opening is formed by placing a die cut piece of 3M General Purpose PTFE Skived Film Tape 5180 (3.5 mil thickness) over the substrate. The exposed surface of the substrate defined by the mask is coated with “Adhesive Formulation C” using a benchtop notch bar coater with the mask providing the coating gap. The adhesive is allowed to air dry and then the mask is removed leaving a patch of a first adhesive layer 30 containing the API as in FIG. 4.

Next, the benchtop notch bar coater (gap setting of 0.008 inches (0.2 mm)) is used to coat “Adhesive Formulation A” over the dried adhesive patch. The coating of “Adhesive Formulation A” as a second adhesive layer 50 completely covers the surface of the dried adhesive patch 30 as well as an area of the substrate beyond the perimeter of the dried adhesive patch as in FIG. 5. The second coating is allowed to air dry. A polyethylene backing layer is laminated to the dried layer of “Adhesive Formulation A” to form a finished drug delivery device 1.

All references and publications cited herein are expressly incorporated herein by reference in their entirety into this disclosure, except to the extent they may directly contradict this disclosure. Illustrative embodiments of this disclosure are discussed, and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1. A method of making an adhesive article, comprising:
applying a first adhesive to a first area of a substrate to form a first adhesive layer, the first adhesive including an active pharmaceutical ingredient;
applying a second adhesive to the first adhesive layer and a second area of the substrate to form a second adhesive layer, wherein the second area of the substrate completely surrounds a perimeter of the first area of the substrate;
cutting or punching a portion of the first and second adhesive layers into a shape that includes all of the first area and at least a portion of the second area surrounding the perimeter of the first area; and
allowing the active pharmaceutical ingredient to diffuse from the first adhesive layer into the second adhesive layer.

2-3. (canceled)

4. The method of claim 1, wherein the cut or punched shape has a surface area of between 5 and 100 cm².

5-6. (canceled)

7. The method of claim 1, further comprising disposing a backing layer adjacent to the second adhesive layer such that the first and second adhesive layers are disposed between the backing layer and the substrate.

8. The method of claim 7, wherein cutting or punching includes cutting through the backing layer to form a desired shape.

9. The method of claim 8, further comprising removing portions of the second adhesive layer and the backing layer that is outside of a perimeter of the desired shape after cutting through the backing layer.

10. The method of claim 9, wherein the concentration of active pharmaceutical ingredient in the removed portions of the second adhesive layer is less than 1% of the concentration of active pharmaceutical ingredient in the first adhesive layer.

11. The method of claim 7, further comprising cutting or punching the substrate to form a liner section comprising a portion that surrounds the first and second adhesive layers and is free of the first and second adhesive layers.

12. The method of claim 1, further comprising disposing an excipient in at least the first adhesive, wherein the excipient is adapted to increase the diffusion rate of the active pharmaceutical ingredient into the second adhesive layer.

13. A method of making an adhesive article, comprising:
applying an active pharmaceutical ingredient to a first area of a substrate;
applying an adhesive to the substrate such that the adhesive overlaps the active pharmaceutical ingredient and is in contact with the substrate in a second area of the substrate;
converting the substrate into a desired shape that includes all of the active pharmaceutical ingredient and at least a portion of the adhesive; and
allowing the active pharmaceutical ingredient to diffuse into at least a portion of the adhesive.

14-17. (canceled)

18. The method of claim 1, wherein the active pharmaceutical ingredient has a molecular weight of less than 500 Daltons.

19. An adhesive article comprising:
an active pharmaceutical ingredient;
a substrate comprising a first surface and a second surface, wherein the second surface comprises a first area and a second area;
a first adhesive layer disposed on the first area of the second surface of the substrate, the first adhesive layer comprising a first surface in contact with the second surface of the substrate and a second surface opposed to the second surface of the substrate, wherein the active pharmaceutical ingredient is present in the first adhesive layer at a first concentration; and
a second adhesive layer comprising a first surface and a second surface opposed to the first surface, wherein a first portion of the first surface of the second adhesive layer is in contact with the second surface of the first adhesive layer and a second portion of the first surface of the second adhesive layer is disposed on the second area of the second surface of the substrate, wherein the active pharmaceutical ingredient in the second portion of the second adhesive layer is present at a second concentration;
wherein the second concentration is less than the first concentration.

20. (canceled)

21. The adhesive article of claim 19, further comprising a backing layer in contact with the second surface of the second adhesive layer.

22. The adhesive article of claim 21, wherein the backing layer has a surface area of between 5 and 100 cm².

23. (canceled)

24. The adhesive article of claim 19, wherein the active pharmaceutical ingredient has a molecular weight of less than 500 Daltons.

25. The adhesive article of claim 19, wherein an outer perimeter of the second area of the second surface of the substrate comprises a circular or ovular shape.

26. The adhesive article of claim 19, wherein the first adhesive layer is configured to be applied to and remain in contact with the skin of a user.

27. The adhesive article of claim 19, wherein the active pharmaceutical ingredient is present in the adhesive article at a therapeutic amount.

28. The adhesive article of claim 19, wherein the active pharmaceutical ingredient has a molecular weight of no more than 600 Daltons.

29. The adhesive article of claim 19, wherein the second surface of the substrate comprises a third area adjacent to the second area, and further wherein the third area defines a perimeter of the adhesive article.

30. The adhesive article of claim 19, further comprising a penetration enhancer.

31. (canceled)
